(19) United States
(12) Patent Application Publication
(10) Pub. No.: US 2005/0148582 A1
(43) Pub. Date: Jul. 7, 2005

(54) Novel composition

(75) Inventors: Philip Christopher Buxton, Harlow (GB); Seona Thomson, Harlow (GB); Dirk Marinus Johannes Van-Schie, Harlow (GB)

Correspondence Address: GLAXOSMITHKLINE;Corporate Intellectual Property - UW2220, P.O. Box 1539, KIng of Prussia, PA, 19406-0939, US

(73) Assignees: SmithKline Beecham p.l.c.; Laboratoire GlaxoSmithKline

(21) Appl. No.: 11/054,837

(22) Filed: Feb. 10, 2005

Related U.S. Application Data
(62) Division of application No. 10/055,817, filed on Jan. 23, 2002.
Application No. 10/055,817 is a continuation-in-part of application No. PCT/GB01/03590, filed on Aug. 8, 2001.

(30) Foreign Application Priority Data
Aug. 8, 2000 (GB) 0019524.8
Aug. 2, 2001 (GB) 0118919.0
Aug. 3, 2001 (GB) 0119022.2
Aug. 7, 2001 (WO) PCT/GB01/03544

(51) Int. Cl. A61K 31/535

(52) U.S. Cl. 514/230.2

(57) ABSTRACT

A pharmaceutical composition comprising N-[(1-ⁿbutyl-4-piperidinyl)methyl]-3,4-dihydro-2H-[1,3]oxazino[3,2-a]indole-10-carboxamide (SB 207266) or a pharmaceutically acceptable salt thereof in combination with one or more pharmaceutically acceptable carriers, wherein at least some of the SB 207266 or salt thereof is in granulated form. Preferably, a filler and/or binder are also present.

This is a continuation-in-part of PCT application No. PCT/GB01/03590 filed 8 Aug. 2001 which claims benefit to PCT Application No. PCT/GB01/03544 filed 7 Aug. 2001, Great Britain patent application 0119022.2 filed 3 Aug. 2001, Great Britain patent application 0118919.0 filed 2 Aug. 2001, and Great Britain patent application 0019524.8 filed 8 Aug. 2000.

This invention relates to a novel composition, for example a tablet or capsule, comprising SB 207266 or a pharmaceutically acceptable salt thereof.

WO 93/18036 (SmithKline Beecham) discloses a large number of condensed indole compounds as 5-HT4 antagonists including, as Example 3 on pages 17-18, N-[(1-ⁿbutyl-4-piperidinyl)methyl]-3,4-dihydro-2H-[1,3]oxazino[3,2-a]indole-10-carboxamide (SB 207266) and its preferred hydrochloride salt (SB 207266-A). These compounds are disclosed for use in the treatment or prophylaxis of gastrointestinal, cardiovascular and CNS disorders, in particular irritable bowel syndrome, and in the treatment of urinary incontinence. WO 93/18036 also states in the general description on pp. 6-7 in general terms that: “Specific cardiac 5-HT₄ receptor antagonists which prevent atrial fibrillation and other atrial arrhythmias associated with 5-HT would also be expected to reduce the occurrence of stroke”. See also U.S. Pat. No. 5,852,014, EP 0 884 319 A2, L. M. Gaster et al, J. Med. Chem., 1995, 38, 4760-4763 and Drugs of the Future, 1997, 22(12), 1325-1332 for the compound SB 207266, which is highly selective for the 5HT₄ receptor over other 5HT receptors. The structure of SB 207266 is as follows:

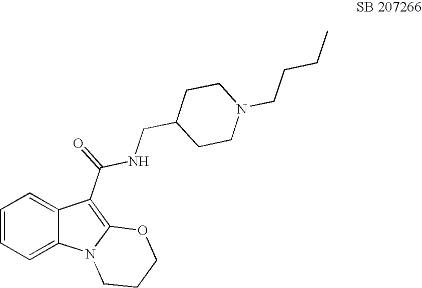

For improved syntheses of SB 207266, see WO 98/07728, WO 98/11067; WO 00/03983; and WO 00/03984.

There are several methods of making the SB 207266 in free base form or as a hydrochloride salt disclosed in the art. Example 3 on page 17-18 of WO 93/18036 discloses the production of SB 207266 in free base form in Methods 1 and 2. Method 2 also discloses conversion to the HCl salt and recrystallisation from ethanol/60-80 petrol to give a white solid. L. Gaster, Drugs of the Future, 1997, 22(12), 1325-1332 discloses a similar method involving HCL salt formation by treatment of SB 207266 free base with anhydrous HCL in ethanol. WO 98/07728 discloses three new methods for making the free base on page 6 line 5 to page 7 line 20. WO 98/07728 also discloses two methods of making the HCl salt (SB 207266-A)—Method A on page 7 line 22 to page 8 line 9, and Method B on page 8 line 10 to page 8 line 19. In page 8 lines 10-19 of WO 98/07728, Method B for making the SB 207266 HCl salt is as follows: “N-[(1-Butyl-4-piperidinyl)methyl]-3,4-dihydro-2H-[1,3]-oxazino[3,2-a]indole-10-carboxamide (SB-207266) (100 g, 0.27 mol) was dissolved in ethanol (870 ml) and the resulting solution filtered to remove particulates. Anhydrous HCl in ethanol (83 ml, 3.6M, 0.30 mol) was added causing the product to precipitate out of solution. The slurry was heated to redissolve the solid and hexane (550 ml) was added. After cooling to room temperature, the mixture was cooled to 0-5° C. and stirred at that temperature for about two hours. The solid was isolated by filtration and dried in vacuo at about 40° C. to give the product, N-[(1-butyl-4-piperidinyl)methyl]-3,4-dihydro-2H-[1,3]-oxazino[3,2-a]indole-10-carboxamide hydrochloride, (102.8 g) in 94% yield.”

### THE INVENTION

It has now been recognised that there are problems with certain processes for making the SB 207266 HCl salt, which processes are similar or identical to the process disclosed as Method B in page 8 lines 10-19 of WO 98/07728 in that the HCl salt is dissolved in ethanol, industrial methylated spirits (IMS, e.g. ethanol containing ca. 1% methanol) or similar and crystallised by addition of a C₅-C₁₀ hydrocarbon (e.g. hexane and/or heptane e.g. n-heptane) and/or a solvent containing a C₅-C₁₀ hydrocarbon (e.g. hexane and/or heptane e.g. n-heptane).

The first aspect of the newly recognised problem is that such processes produce the SB 207266 hydrochloride salt in the form of particles of extremely small particle size. For example, the following Table 1 shows the particle size data from batches of the HCl salt (SB-207266-A) made using a process similar to Method B of page 8 of WO 98/07728 but using IMS instead of ethanol and n-heptane instead of hexane in the crystallisation step (this process is disclosed in Description 1 hereinafter):

TABLE 1

| Batch | DV 90 (μm) | DV 50 (μm) | DV 10 (μm) |
|---|---|---|---|
| BDC-H-01C | 12.8 | 5.3 | 1.4 |
| BDC-G-02C | 13.8 | 5.7 | 1.5 |
| BDC-G-03C | 16.4 | 6.8 | 1.8 |
| BDC-G-04C | 14.4 | 5.3 | 1.4 |
| BDC-G-05C | 14.6 | 5.8 | 1.5 |
| Average | 14.4 | 5.8 | 1.5 |

DV 90, DV 50 and DV 10 respectively mean that 90%, 50% and 10% by volume of the material is less than the micron size specified.

The second aspect of the newly recognised problem, is the discovery that the SB 207266 HCl salt produced by these processes is very cohesive and has poor flowability/flow characteristics.

The third aspect of the newly recognised problem is that at above certain concentrations in a pharmaceutical formulation, this cohesive drug material causes the composition to be sufficiently poorly-flowing that it cannot easily be tabletted or made into capsules, when the SB-207266 HCl salt is in combined with standard methylcellulose, mannitol and Mg stearate excipients. It has been found that a composition for SB 207266, for human oral administration, containing: SB-207266 HCl salt (ca. 5.0 mg), Microcrystalline cellulose (30.0 mg), Mannitol (112.0 mg), Mg Stearate (3.0 mg), with total tablet weight=ca. 150 mg (=Comparative Example 1 below), is possible to tablet. However, higher concentrations of the SB-207266 HCl salt are not easily tabletted using this type of formulation. This is particularly relevant as the clinical maintenance dose for treatment or prophylaxis of atrial fibrillation is now thought likely to be about 20, 50 or 80 mg/day (see the clinical protocol in Example 8 hereinafter and the tablets of Examples 4, 5, 6 and 7), whereas the clinical doses tried previously for treatment of irritable bowel syndrome were only 0.05, 0.25, 1 and 5 mg/day.

The fourth aspect of the newly recognised problem is that the small-particle size SB-207266 HCl salt has a low bulk density, densifying on the addition of water. This means that less material can be added to a mixer of fixed volume, leading to a less efficient manufacturing process as large volumes of equipment have to be used for relatively small volumes of drug (smaller throughput in plant).

It has now been discovered that some or all of these problems can be at least partly overcome or mitigated by the forming the SB 207266 HCl salt into granules which have a particler size larger than than of the original SB 207266 HCl salt, e.g. by using a wet granulation process. These granules are found to have better flow characteristics for e.g. tabletting purposes. It has also been found that the incorporation of a filler into the granules, especially an insoluble filler such as CaHPO₄ and/or Ca₃(PO₄)₂, can help to form granules with pharmaceutically advantageous properties, e.g. often minimising dissolution of the very soluble SB 207266 HCl salt in the granulation solvent and so minimising undesirable fusion of granules after removal of the solvent. Some or all of these advantages are also expected to be gained for the free base which is believed also to have usually a small-particle size, e.g. the free base is very slow to filter when crystallised by the addition of hexane to a toluene solution (e.g. as in Method A on page 6 lines 19-23 and Method C on page 7 lines 14-20 of WO 98/07728). Similarly salts other than the HCl salt are thought to benefit too.

Therefore, a first aspect of the invention provides a pharmaceutical composition comprising N-[(1-ⁿbutyl-4-piperidinyl)methyl]-3,4-dihydro-2H-[1,3]oxazino[3,2-a]indole-10-carboxamide (SB 207266) or a pharmaceutically acceptable salt thereof in combination with one or more pharmaceutically acceptable carriers, wherein at least some of the SB 207266 or salt thereof is in granulated form.

Preferably, substantially all or all of the SB 207266 or salt thereof is in granulated form.

Preferably the composition is a tablet, or the invention can be a capsule containing said composition. Preferably, 50% or more by weight or by volume of the granules including the SB 207266 or salt thereof have a particle size of: ≧100 microns (micrometres) e.g. 100 to 1000 microns, more preferably ≧200 microns e.g. 200 to 1000 or 200 to 500 microns, still more preferably ≧250 microns e.g. 250 to 500 microns. In other words, this means that preferably the granules including the SB 207266 or salt thereof have particle size defined by a particle size defined by a “D50”, or median particle size, e.g. by weight (DM50) or by volume (DV50), of ≧100 microns or one of the other above-specified preferred size ranges.

Preferably, 90% or more by weight or by volume of the granules including the SB 207266 or salt thereof have a particle size of: ≧10 microns (micrometres) e.g. 10 to 1000 microns, more preferably ≧50 microns e.g. 50 to 1000 or 50 to 500 microns, still more preferably ≧100 microns e.g. 100 to 500 microns. In other words, this means that preferably the granules including the SB 207266 or salt thereof have a particle size defined by a “D10”, e.g. by weight (DM10) or by volume (DV10), of ≧10 microns or one of the other above-specified preferred size ranges. As an alternative definition, preferably 10% or less by weight or by volume of the granules including the SB 207266 or salt thereof have a particle size of: ≦10 microns (micrometres), more preferably ≦50 microns, still more preferably ≦100 microns.

Compositions of the invention containing granules with the above-mentioned medium to large particle sizes are generally less cohesive, flow better, and are thus less likely to cause the above-mentioned formulation problems.

Preferably, 50% or more by weight or by volume of the particles of the SB 207266 or salt thereof (e.g. before forming into granules and/or after granule formation; e.g. within the granules) have a particle size of ≦80 microns (micrometres), more preferably ≦50 microns, still more preferably ≦20 microns, even more preferably ≦10 microns, most preferably ≦8 microns. In other words, this means that preferably the particles of the SB 207266 or salt thereof (e.g. before forming into granules and/or after granule formation; e.g. within the granules) have a particle size defined by a “D50”, or median particle size, e.g. by weight (DM50) or by volume (DV50), of ≦80 microns or ≦50 microns or one of the other above-specified preferred size ranges.

Preferably, 10% or more by weight or by volume of the particles of the SB 207266 or salt thereof (e.g. before forming into granules and/or after granule formation; e.g. within the granules) have a particle size of ≦20 microns (micrometres), more preferably ≦10 microns, still more preferably ≦5 microns, even more preferably ≦2.5 microns, most preferably ≦2 microns. In other words, this means that preferably the particles of the SB 207266 or salt thereof (e.g. before forming into granules and/or after granule formation; e.g. within the granules) have a particle size defined by a “D10”, e.g. by weight (DM10) or by volume (DV10), of ≦20 microns or one of the other above-specified preferred size ranges.

Preferably, 90% or more by weight or by volume of the particles of the SB 207266 or salt thereof (e.g. before forming into granules and/or after granule formation; e.g. within the granules) have a particle size of ≦100 microns (micrometres), more preferably ≦50 microns, still more preferably ≦20 microns. In other words, this means that preferably the particles of the SB 207266 or salt thereof (e.g. before forming into granules and/or after granule formation; e.g. within the granules) have a particle size defined by a “D90”, e.g. by weight (DM90) or by volume (DV90), of ≦100 microns, more preferably ≦50 microns, still more preferably ≦20 microns.

As discussed above, SB 207266 or salts with such small particle sizes are the ones most likely to give the problems above-mentioned, and are most likely to benefit from the present invention.

In general, particle sizes (D50, D10, D90, et al.) can be measured by sieving with one or more sieves (e.g. for granules before further processing into tablets, and/or for measuring the powder inside capsules).

Alternatively, particle sizes can be measured by laser diffraction, also known as low angled laser light scattering (LALLS). Laser diffraction is based on the angular distribution of scattered light. Laser diffraction is known to the skilled person and can use an algorithm based on a Fraunhoefer or Mie optical model also known to the skilled person. Further details of the laser diffraction technique can be found in: Clive Washington “Particle Size Analysis in Pharmaceutics and Other Industries, Theory and Practice”, Ellis Horwood Limited, 1992, see in particular Chapter 6, p. 109-133, details of which are hereby incorporated by reference. The Fraunhoefer calculation is described therein and is commonly performed by the software analysis package provided as part of commercially available laser diffraction apparatus e.g. as now described. Suitable laser diffraction apparatus include (a) the Malvern Mastersizer S, obtainable from Malvern Instruments Limited, Enigma Business Park, Grovewood Road, Malvern, Worcestershire WR14 1XZ, United Kingdom, email: www.malvern.co.uk; and (b) the Sympatec HELOS/QUIXEL, obtainable from Sympatec UK and Ireland, Bury Business Centre, Kay Street, Bury BL96BU, United Kingdom, email: sympatec.uk@btinternet.com.

Alternatively, particle sizes can be measured directly, (for example optically e.g. by microscope, or otherwise), particularly in a tablet. For example, particle sizes can be so measured in a section through the tablet (for example obtained by breaking a tablet into 2 pieces and observing the cross-sectional face); diameters of specific particles can be measured which enables an estimation of the particle size distibution by volume and thence by weight.

Particle size analysis methods typically assume sphericity of particles in the calculation of the distribution. In cases where non-sperical particles are analysed, skilled interpretation is required to understand the influence that shape may have on skewing the size distribution. Particle sizing techniques that utilise images of the particles such as microscopy can, however, accurately infer particle shape and size, though typically size would still be expressed assuming sphericity.

Preferably, the SB 207266 or salt thereof (e.g. the HCl salt) is of a form obtainable by, e.g. preferably is made by, a process in which the SB 207266 or salt (e.g. HCl salt) is dissolved in ethanol or an ethanol-containing solvent such as industrial methylated spirits (IMS, e.g. ethanol containing ca. 1% methanol) to form a solution and is crystallised from the solution by addition of a C₅-C₁₀ hydrocarbon (e.g. hexane and/or heptane e.g. n-heptane) and/or a solvent containing a C₅-C₁₀ hydrocarbon (e.g. hexane and/or heptane e.g. n-heptane). Such processes often form SB 207266 or salts with small particle sizes—at least for the HCl salt—which products are most likely to give the problems above-mentioned, and are which most likely to benefit from the present invention.

Preferably, the SB 207266 or salt thereof is present in the composition in at least 3.5 weight %, more preferably in at least 4 weight % or at least 4.4 weight % or at least 5 weight % or at least 6 weight % or at least 8 weight %, by weight of the composition. Preferably, the SB 207266 or salt thereof is present in the composition in up to 95 weight %, more preferably up to 70 weight %, most preferably up to 50 weight %. For example, about 10-100 mg (e.g. 10, 20, 25, 30, 40, 50, 75, 80 or 100 mg) of SB 207266 or salt thereof (measured either as the free base or as the actual weight including counterions) for every 250 mg of weight of composition (e.g. for every 250 mg coated or uncoated tablet weight) is ideal.

Preferably, the N-[(1-ⁿbutyl-4-piperidinyl)methyl]-3,4-dihydro-2H-[1,3]oxazino[3,2-a]indole-10-carboxamide (SB 207266) or a pharmaceutically acceptable salt thereof comprises (e.g. is) the hydrochloride salt of SB 207266 (SB 207266-A).

Preferably, the granules containing the SB 207266 or salt thereof also contain a filler (diluent). Mixing the filler with the SB 207266 or salt thereof before granulation often aids formation of granules. Granulating pure SB 207266 or a salt is difficult.

Preferably, the filler (diluent) is abrasive. This helps to aleviate the cohesiveness of the SB 207266 or salt, and aids the flowability of the granules.

Preferably, the filler is brittle (as opposed to elastic or plastic). Brittleness can be determined by tests known to the skilled man such as compaction simulation tests which for example determine Young's modulus of the filler. Preferably, the filler (diluent) is insoluble, practically insoluble, very slightly soluble or slightly soluble (more preferably insoluble or practically insoluble) in a/the granulating solvent, e.g. water and/or ethanol and/or isopropanol. The terms “practically insoluble”, “very slightly soluble” and/or “slightly soluble” can be as defined in the British Pharmacopoeia, the European Pharmacopoeia and/or the US Pharmacopoeia. “Practically insoluble” according to the British Pharmacopoeia 1999 (page 11) means that at least 10 litres of the solvent is required to dissolve 1 gram of the filler/solute (e.g. at ambient temperature, e.g. 15 or 20 or preferably 25° C.). “Very slightly soluble” according to the British Pharmacopoeia means that at least 1 litre and up to 10 litres of the solvent is required to dissolve 1 gram of the filler/solute (e.g. at 25° C.). “Slightly soluble” according to the British Pharmacopoeia means that at least 100 ml and up to 1 litre of the solvent is required to dissolve 1 gram of the filler/solute (e.g. at 25° C.). The insoluble, practically insoluble, very slightly soluble or slightly soluble (preferably insoluble or practically insoluble) fillers form a surface or substrate for the SB 207266 free base or salt to adhere to during wet granulation. This minimises/reduces undesirable fusion of and/or excessive binding between all the ingredients of the formulation after removal of the granulation solvent, and in particular reduces excessive binding of drug to other intragranular excipients. This improves the quality of the granules, and usually increases the rate of dissolution of the final composition (e.g. tablet).

In contrast, although soluble fillers such as lactose or mannitol are possible, they are more likely to dissolve at least partly during wet granulation, often causing fusion problems in the final granules.

Thus tests using the soluble filler lactose and SB207266 hydrochloride present during wet (water) granulation have been found to lead to less superior tablets in which the drug and filler are more intertwined and more tightly bound together, so that the disintegration time is longer than that of the corresponding tablet made using the insoluble filler CaHPO₄.

Preferably, the filler comprises any pharmaceutically acceptable metal (e.g. calcium or magnesium) salt which is insoluble, practically insoluble, very slightly soluble or slightly soluble (preferably insoluble) in the granulating solvent e.g. water and/or ethanol. The salt can for example be a phosphate, hydrogen phosphate, carbonate or hydrogen carbonate salt. Such insoluble-to-slightly soluble salts include calcium phosphate, dibasic calcium phosphate, calcium carbonate, magnesium carbonate, magnesium phosphate, etc.

Preferably, the filler comprises dibasic calcium phosphate (i.e. dicalcium phosphate, calcium hydrogen phosphate, CaHPO₄), more preferably dibasic calcium phosphate hydrate e.g. dihydrate (i.e. CaHPO₄.2H₂O). Anhydrous dibasic calcium phosphate can also be used. CaHPO₄, e.g. hydrated or anhydrous, is abrasive and helps to aleviate the cohesiveness of the SB 207266 or salt; and it is insoluble in water which helps the granulation process as described above. Alternatively or additionally, the filler can comprise calcium phosphate, i.e. tribasic calcium phosphate, Ca₃(PO₄)₂. Preferably, a fine grade filler e.g. fine grade CaHPO₄ (such as Calipharm™, as disclosed e.g. in the Handbook of Pharmaceutical Excipients, 3rd edn, 2000) or fine grade Ca₃(PO₄)₂ is used.

The filler is preferably present in up to 95% by weight of the granules, and/or up to 85% or up to 70% by weight of the composition. Preferably, the filler is present in ≧15 wt % or ≧20 wt % or ≧30 wt % of the composition. For example, the filler is preferably present in from 15 to 85% or from 15 to 70% by weight of the composition. Preferably, the weight ratio of filler to drug in the composition or granules is at least 1:3, preferably at least 1:2.5 or at least 1:2 or at least 2:3.

Preferably, the composition includes an excipient which acts as a compression and/or granulation aid, for example comprising or being microcrystalline cellulose (MCC). The compression and/or granulation aid is preferably present in at least 15 wt %, more preferably 15-50 wt % or 15-30 wt % (e.g. about 20 wt %) of the composition. Preferably, the compression and/or granulation aid comprises MCC having a nominal mean particle size of about 25 μm to about 150 μm, more preferably about 50 μm to about 100 μm. Suitable grades of MCC include Avicel PH-102 (100 μm mean particle size) and Avicel PH-101 (50 μm mean particle size) available from FMC Corporation.

“Compression aid” means an excipient which aids in overall compressibility. For example, MCC acts to help plastic deformation when tabletting.

“Granulation aid” means an excipient which helps to disperse the granulating solvent to an extent during granulation; MCC does this to an extent. MCC also helps to determine the end-point of wet granulation (i.e. at what point sufficient granulation solvent e.g. water has been added) because it is water-adsorbent but practically insoluble in water, so it does not dissolve substantially in water if too much water is added as granulation solvent. Therefore, preferably, the compression and/or granulation aid is insoluble or practically insoluble (e.g. as defined above) in the granulation solvent e.g. in water.

The compression and/or granulation aid can be present inside the granules (i.e. intragranular) and/or outside the granules (i.e. extragranular). Preferably, the compression and/or granulation aid is present inside the granules of the composition (intragranular) (which does not exclude the possibility that a portion of the binder is present outside the granules).

Preferably, the composition includes a binder. The binder acts to bind the drug (SB207266 or a salt thereof) onto the other intragranular ingredients, increasing the strength of the granules so that for example when compressed they form stronger bonds. The binder is preferably a cellulosic binder for example comprising or being hydroxypropylmethylcellulose (HPMC) (e.g. low viscosity HPMC such as Pharmacoat 603, made by Shinogi, Japan). Other possible cellulosic binders can include hydroxypropylcellulose (HPC), hydroxyethylcellulose (HEC), hydroxymethylcellulose (HMC), methyl cellulose (e.g. low to medium viscosity), ethyl cellulose, etc. Another suitable binder includes povidone (polyvinylpyrollidone, PVP; this is an essentially linear, non-crosslinked polymer, see Handbook of Pharmaceutical Excipients, 3rd edn, 2000), for example K30, K60 or K90 grade povidone and/or povidone having about 50,000 to about 1,000,000 molecular weight. The binder can preferably be present in about 1 to about 10 weight % of the composition, for example about 2.5 to about 10 weight % or about 1 to about 5 weight % (e.g. about 5 wt %) of the composition. HPMC is preferably present in about 5 wt %. The binder is preferably present in the granules (i.e. is intragranular) (which does not exclude the possibility that a portion of the binder is present outside the granules).

Preferably, the binder is soluble, freely soluble or very soluble in the granulation solvent, e.g. in water, ethanol and/or isopropanol, preferably water. “Soluble” according to the British Pharmacopoeia 1999 means that from 10 to 30 ml of the solvent is required to dissolve 1 gram of the solute at ambient temperature (e.g. 15 to 25° C.). “Freely soluble” according to the British Pharmacopoeia means that from 1 to 10 ml of the solvent is required to dissolve 1 gram of the solute (e.g. at 25° C.). “Very soluble” according to the British Pharmacopoeia means that less than 1 ml of the solvent is required to dissolve 1 gram of the solute (e.g. at 25° C.).

Preferably, the composition includes a disintegrant (e.g. tablet disintegrant) such as sodium starch glycollate (e.g. Primojel or Explotab™), croscarmellose sodium (e.g. Ac-Di-Sol™), or crospovidone (cross-linked polyvinylpyrollidone). The disintegrant can be preferably present in about 1 to about 10 weight % of the composition, for example about 2.5 to about 10 weight % or about 1 to about 5 weight % (e.g. about 5 wt %) of the composition. Sodium starch glycollate is preferably present in about 5 wt %. Preferably, the disintegrant is present outside the granules (extragranular) (which does not exclude the possibility that a portion of the disintegrant is present inside the granules).

Preferably, the composition includes a lubricant, for example comprising or being an alkaline earth metal stearate such as magnesium stearate. The lubricant can be present in preferably about 0.2 to about 5 weight % or more preferably about 0.2 to about 2 weight % (e.g. about 1 wt %) of the composition. Preferably, the lubricant is present outside the granules (extragranular) (which does not exclude the possibility that a portion of the lubricant is present inside the granules).

Preferably, the granules (i.e. the intragranular ingredients) form ≧70%, ≧80%, ≧85%, ≧90% or ≧93% by weight of the composition, for example about 94 wt % (e.g. as in Examples 4-7). That is, preferably, the extragranular ingredients form ≦30%, ≦20%, ≦15%, ≦10% or ≦7%, for example about 6%, by weight of the composition.

A second aspect of the invention provides a method (process) of making a pharmaceutical composition comprising N-[(1-ⁿbutyl-4-piperidinyl)methyl]-3,4-dihydro-2H-[1,3]oxazino[3,2-a]indole-10-carboxamide (SB 207266) or a pharmaceutically acceptable salt thereof in combination with one or more pharmaceutically acceptable carriers,
the method (process) comprising forming at least some of the SB 207266 or salt thereof into granules.

Preferably, the method also comprises mixing some or all of the SB 207266 or salt thereof with a filler (diluent), and optionally a binder and/or a compression and/or granulation aid, before granulation. The filler, binder and/or compression and/or granulation aid can be as defined herein.

Preferably, the granules are formed in the presence of a granulating solvent (i.e. using a “wet granulation” process). Preferably the granulating solvent comprises or is water and/or ethanol and/or isopropanol, preferably water. The solvent can be added after mixing of the SB 207266 or salt with the filler and/or binder. Preferably, just sufficient solvent to enable granulation is used, typically about 15% to about 20% v/w, e.g. 17% or 20% v/w.

Preferably, the solvent is removed after formation of the granules, e.g. by drying. Fluid bed drying is preferred.

Preferably, the filler (diluent) is insoluble, practically insoluble, very slightly soluble or slightly soluble in the granulation solvent, e.g. as defined herein.

Preferably, after formation the granules (e.g. the dried granules and/or the wet granules) are milled to a particle size suitable for use in tablets or capsules, e.g. using a comminuting mill (e.g. for dry granules). For example, the granules can be milled such that they pass through seive or screen with a ≦0.055 inch (1.40 mm) or ≦0.032 inch (0.81 mm) hole size. The granules can be passed through such a seive or screen during or after milling.

Preferably, (after formation of the granules) the granules are then (i) optionally mixed with other pharmaceutically acceptable excipient(s) and (ii) compressed into tablets or filled into capsules. Such extragranular excipient(s) preferably include a disintegrant and/or a lubricant and/or a compression aid, e.g. as defined herein.

A third aspect of the invention provides a method of making a pharmaceutical composition comprising N-[(1-ⁿbutyl-4-piperidinyl)methyl]-3,4-dihydro-2H-[1,3]oxazino[3,2-a]indole-10-carboxamide (SB 207266) or a pharmaceutically acceptable salt thereof in combination with one or more pharmaceutically acceptable carriers,
the method comprising:
(a) dissolving the SB 207266 or salt thereof in ethanol or an ethanol-containing solvent such as industrial methylated spirits (IMS, e.g. ethanol containing ca. 1% methanol) to form a solution,
(b) crystallising the SB 207266 or salt thereof from the solution by addition of a C₅-C₁₀ hydrocarbon (e.g. hexane and/or heptane) and/or a solvent containing a C₅-C₁₀ hydrocarbon (e.g. hexane and/or heptane), and
(c) forming at least some of the SB 207266 or salt thereof into granules.

In this third aspect of the invention, it is particularly preferable that the SB 207266 or salt thereof comprises (e.g. is) the hydrochloride salt of SB 207266.

Preferably, the method of the third aspect of the invention comprises the additional step after formation of the granules of (d) mixing the granules with other pharmaceutically acceptable excipient(s) and compressed into tablets or filled into capsules.

SB 207266 or the salt thereof may conveniently be administered by any of the routes conventionally used for drug administration, for instance, parenterally, orally, topically or by inhalation.

Procedures for making the composition and/or tablet and/or capsule may involve mixing, granulating and compressing the ingredients as appropriate to the desired preparation.

The excipient(s)/carriers used in the composition should be “pharmaceutically acceptable” in the sense of being compatible with the other ingredients of the formulation and not deleterious to the recipient thereof.

The pharmaceutically acceptable carrier employed may be, for example, a solid. Exemplary of solid carriers are lactose, terra alba, sucrose, talc, gelatin, agar, pectin, acacia, magnesium stearate, stearic acid and the like. Similarly, the carrier may include time delay material well known to the art, such as glyceryl mono-stearate or glyceryl distearate alone or with a wax.

A wide variety of pharmaceutical forms can be employed. Thus, if a solid carrier is used, the preparation can be tableted, placed in a hard gelatin capsule in powder or pellet form or in the form of a troche or lozenge. The amount of solid carrier will vary widely but preferably will be from about 25 mg to about 1 g.

A particularly preferred oral composition for SB 207266, for human oral administration, is as follows:

| | |
|---|---|
| SB-207266 | 5.0 mg |
| Microcrystalline cellulose | 50.0 mg |
| HPMC | 12.5 mg |
| Sodium Starch glycollate | 12.5 mg |
| Dicalcium phosphate | 167.5 mg |
| Mg stearate | 2.5 mg |
| | 250 mg |

HPMC = hydroxypropylmethylcellulose

The dose in the above composition can readily be increased to 20 mg. This composition is the result of a granulation process.

These and other suitable oral compositions for SB 207266 are described in the Examples hereinbelow.

### UTILITY/INDUSTRIAL APPLICATION

The pharmaceutical composition of the present invention containing SB 207266 or a salt thereof can be used in the treatment or prophylaxis of atrial arrhythmias such as atrial fibrillation (AF), and/or in the treatment or prophylaxis of atrial remodelling. Atrial fibrillation is preferred. In particular, it is thought that compositions such as tablets containing SB 207266 or a salt thereof can be administered to patients with symptomatic persistent atrial fibrillation (AF) in order to inhibit symptomatic recurrences of atrial fibrillation in these patients. A proposed clinical protocol is given in Example 8 hereinafter.

Therefore the invention also provides a method of treatment or prophylaxis of atrial arrhythmia, such as atrial fibrillation, compising administering to a mammal (e.g. human) in need of such treatment or prophylaxis an effective amount of a pharmaceutical composition as defined herein. The invention also provides a method of inhibiting symptomatic recurrences of atrial fibrillation in a mammal (e.g. human) with symptomatic persistent atrial fibrillation compising administering to the mammal an effective amount of a pharmaceutical composition as defined herein.

SB 207266 compositions might also reduce the occurrence of stroke in AF patients. SB 207266 compositions might also be useful in the treatment and/or prophylaxis of urinary incontinence.

All publications, including but not limited to patents and patent applications, cited in this specification are herein incorporated by reference as if each individual publication were specifically and individually indicated to be incorporated by reference herein as though fully set forth.

The invention will now be described by reference to the following Examples which are merely illustrative and are not to be construed as a limitation of the scope of the present invention.

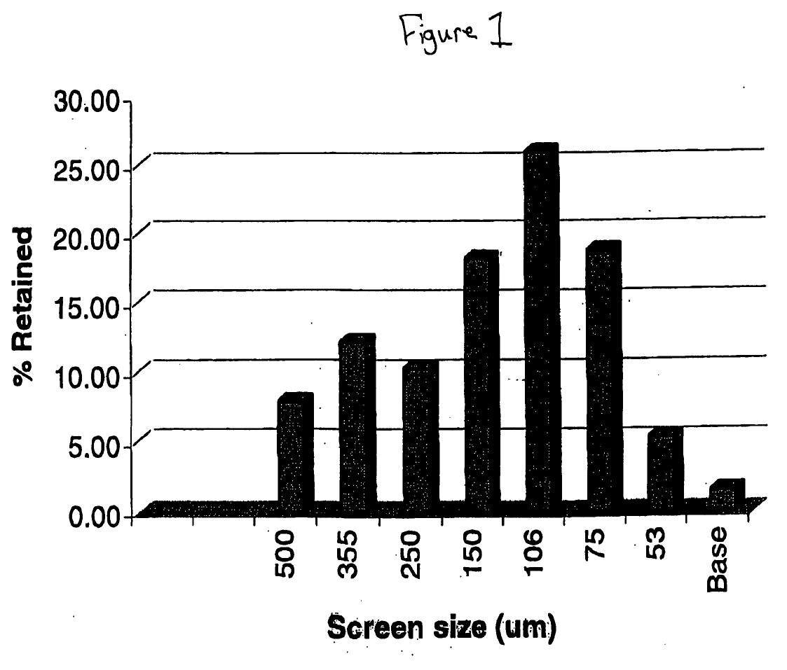

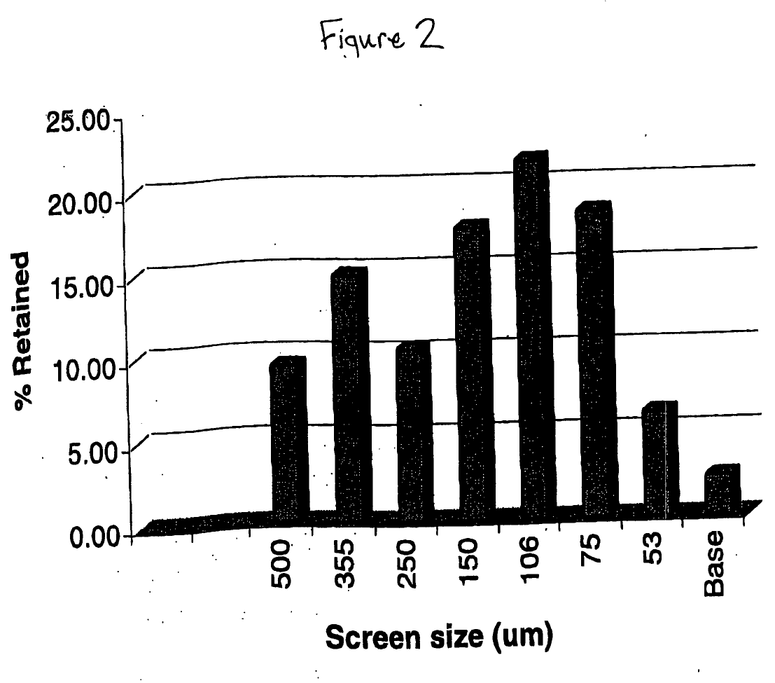

FIGS. 1 and 2 show a particle size analysis of the granules produced by Examples 6 and 7 respectively after milling but before blending with extragranular excipients and tabletting.

### EXAMPLES

SB 207266-N-[(1-ⁿbutyl-4-piperidinyl)methyl]-3,4-dihydro-2H-[1,3]oxazino[3,2-a]indole-10-carboxamide—is made using the synthetic methods decribed in the introduction, i.e. as described in one or more of WO 98/07728, WO 98/11067; WO 00/03983; and/or WO 00/03984.

For a method of making the SB 207266 hydrochloride salt from the free base, see in particular Method B in page 8 lines 10-19 of WO 98/07728 and minor variations thereof which are described in full in the “Introduction” and “The Invention” sections above (e.g. see page 2 hereinabove). One minor and wholly equivalent variation of the WO 98/07728 Method B is given in detail in the following Description 1, in which IMS is used instead of ethanol and n-heptane is used instead of hexane in the crystallisation step. IMS is industrial methylated spirits, and in Description 1 the specific type of IMS used was ethanol containing ca. 1% methanol.

Description 1

N-[(1-Butyl-4-piperidinyl)methyl]-3,4-dihydro-2H-[1,3]-oxazino[3,2-a]indole-10-carboxamide (SB-207266) (10 g, 0.27 mol) was dissolved in IMS (825 ml) and the resulting solution filtered to remove particulates. Anhydrous HCl in IMS (174 ml, 1.7M, 0.29 mol) was added causing the product to precipitate out of solution. The slurry was heated to redissolve the solid and n-heptane (550 ml) was added. After cooling to room temperature, the mixture was cooled to 0-5° C. and stirred at that temperature for about one hour. The solid was isolated by filtration and dried in vacuo at about 40° C. to give the product, N-[(1-butyl-4-piperidinyl)methyl]-3,4-dihydro-2H-[1,3]-oxazino[3,2-a]indole-10-carboxamide hydrochloride, (98 g) in 89% yield.

### Examples 1, 2, 3, 3A, 4 and 5

### SB 207266 Pharmaceutical Compositions

### Comparative Example 1

An oral composition for SB 207266, for human oral administration, is as follows:

| | |
|---|---|
| SB-207266 | 5.0 mg |
| Microcrystalline cellulose | 30.0 mg |
| Mannitol | 112.0 mg |
| Mg Stearate | 3.0 mg |
| Tablet weight | 150 mg |

This composition is not in accordance with the present invention.

### Example 2

An oral composition for SB 207266, for human oral administration, according to the present invention, is as follows:

| | |
|---|---|
| SB-207266 | 5.0 mg |
| Microcrystalline cellulose | 50.0 mg |
| HPMC (hydroxypropylmethylcellulose) | 12.5 mg |
| Sodium Starch glycollate | 12.5 mg |
| Dicalcium phosphate | 167.5 mg |
| Mg stearate | 2.5 mg |
| Tablet weight | 250 mg |

The dose in this composition can readily be increased to 20 mg. This composition is the result of a granulation process.

### Example 3

The tablet of Example 2 can be varied by increasing the dose of SB 207266 from 5 mg to up to 20, 60, 75, 80 or 100 mg (measured as the free base), and by decreasing the amount of dicalcium phosphate accordingly while keeping the 250 mg tablet weight constant. The composition can use SB 207266 as the free base or as the hydrochloride salt.

### Example 3A

The compositions of Examples 2 and 3 can use either SB 207266 as the free base or as the hydrochloride salt.

### Example 4

### SB-207266-A Tablets with 10, 25, and 40 mg Strength (Measured as Pure Free Base)

Tablets containing the hydrochloride salt of SB 207266 (SB 207266-A) in amounts of 10, 25 or 40 mg (measured as the free base) were made according to the composition in the table below.

Example 4 composition

| | | Quantity (mg/tablet) | | |
|---|---|---|---|---|
| | | 10 mg | 25 mg | 40 mg |
| | | tablet | tablet | tablet |
| Ingredient | Function | strength | strength | strength |
| Active Ingredient | | | | |
| SB-207266-A (hydrochloride) | API | 11.0* | 27.5* | 44.0* |
| Other Ingredients | | | | |
| Microcrystalline Cellulose | Compression | 50.0 | 50.0 | 50.0 |
| (e.g. Ph. Eur. or NF) (e.g. | & | | | |
| Avicel PH-102) | granulation | | | |
| | aid | | | |
| Hydroxypropylmethyl | Binder | 12.5 | 12.5 | 12.5 |
| cellulose (e.g. USP) | | | | |
| (e.g. Pharmacoat 603) | | | | |
| Sodium starch glycollate (e.g. | Disintegrant | 12.5 | 12.5 | 12.5 |
| NF or Ph Eur) | | | | |
| Calcium hydrogen phosphate | Major | 161.5 | 145.0 | 128.5 |
| dihydrate | diluent | | | |
| (Dibasic Calcium Phosphate | | | | |
| dihydrate) (e.g. Ph. Eur. or | | | | |
| USP) (e.g. Calipharm ™) | | | | |
| Magnesium Stearate (e.g. Ph. | Lubricant | 2.5 | 2.5 | 2.5 |
| Eur. or NF) | | | | |
| Purified Water** (e.g. Ph. | Granulating | ** | ** | ** |
| Eur. or USP) | solvent | | | |
| Opadry White YS-1-7003 | Film Coat | 6.25 | 6.25 | 6.25 |
| Purified Water** | | ** | ** | ** |
| Total Tablet Weight | | 256.25 | 256.25 | 256.25 |

*Equivalent to 10, 25, 40 mg respectively of pure free base
**Removed during processing

The SB-207266-A tablets of Example 4 are packed into high density polyethylene (HDPE) bottles with plastic, child-resistant, induction seal caps.

The formulation used a wet granulation process using an insoluble major excipient, Dibasic calcium Phosphate dihydrate (or Dicalcium phosphate). Dibasic calcium Phosphate dihydrate is the major diluent together with microcrystalline cellulose which is added to disperse the granulating solvent and to aid in the overall compressibility. The binding agent added is hydroxypropylmethyl cellulose and the granulation is carried out in a conventional mixer granulator. The granule mix is dried, screened and mixed with sodium starch glycollate as a disintegrant and magnesium stearate as a lubricant to form the compression mix. Tablets are produced on a suitable rotary tablet press, and can be either oval or round in shape.

### Example 4

### Detailed Manufacturing Process, In-Process Controls, and Assembly Process

SB-207266-A, microcrystalline cellulose, dibasic calcium phosphate dihydrate, and hydroxypropylmethyl cellulose are blended together. Purified water is added to the blended powders while mixing in a high shear mixer-granulator. The granules are dried in a fluid bed drier and are then transferred to a mixer, where they are blended with sodium starch glycollate and magnesium stearate. The lubricated mix is compressed into tablet cores using a rotary tablet press. The tablet cores are film coated using an aqueous dispersion of Opadry White YS-1-7003.

Procedure:

1.0 Granulation.

1.1 Blend the SB-207266, microcrystalline cellulose, hydroxypropylmethyl cellulose and dibasic calcium phosphate dihydrate in a suitable high shear mixer-granulator.
1.2 Add the purified water to effect the granulation.
1.3 Dry the granules in a fluid bed drier.
1.4 Pass the dried granules through a stainless steel screen using a suitable mill.
   (For example: the dried granules can be passed through a stainless steel screen during milling using a comminuting mill. A comminuting mill commonly comprises a frustoconical screen forming the wall of the mill and a coaxial axially-rotatable frustoconical impeller closely-spaced to the screen to crush granules poured into the gap between screen and the rotating impeller. Once crushed to the necessary size, the granules can escape through the holes in the screen. The screen can for example have a 0.055 inch or 0.032 inch hole size).
1.5 Determine the yield of the granules.
   2.0 Manufacture of Compression Mix.
2.1 Blend the required quantities of sodium starch glycollate and magnesium stearate with the dried granules
2.2 Determine the yield of compression mix.
   3.0 Tablet Compression.
3.1 Transfer the compression mix to a suitable tablet machine.
3.2 Compress the tablets.
3.3 Determine the yield of the compressed tablets.
   4.0 Film Coating.
4.1 Transfer the tablet cores to a suitable coating machine.
4.2 Rotate the cores and spray on aqueous dispersion of Opadry.
4.3 Release test samples are taken randomly from the batch and appropriately labelled.
   5.0 Bottle Filling
5.1 HDPE bottles are filled to the appropriate fill count, induction sealed and fitted with a child resistant cap using suitably automated equipment.

### Example 5

In a modification of Example 4, formulations containing 20 mg, 30 mg, 50 mg, 75 mg, 80 mg or 100 mg of SB-207266 per tablet (present as the hydrochloride salt, but the dose stated here being measured as the free base) can been used to make tablets; instead of the 10, 25 and 40 mg per tablet amounts given in Example 4. These formulations maintain (a) the total coated tablet weight of 256.25 mg, (b) the total pre-coating tablet weight of 250 mg and (c) the other excipient amounts in the Example 4 compositions, but adjust the amount of Dibasic Calcium Phosphate dihydrate used as the amount of SB 207266 varies. These tablets can be round or oval.

### Example 6

The tablet of Example 5 containing 75 mg of SB 207266 (measured as free base) was modified by changing the percentage of HMPC binder from 5% to 2% by weight of the formulation by lowering the wt % of calcium hydrogen phosphate. The ingredients list follows (the batch made 10 tablets, i.e. 2500 g dry weight):

| | Where? | mg/tablet | weight % |
|---|---|---|---|
| SB-207266 hydrochloride | intragranular | 83.3 mg | 33.3 |
| Microcrystalline cellulose (Avicel | intragranular | 50.0 mg | 20.0 |
| PH-102) | | | |
| HPMC (Pharmacoat 603) | intragranular | 5.0 mg | 2.0 |
| Calcium hydrogen phosphate | intragranular | 96.75 mg | 38.7 |
| (Calipharm) | | | |
| Sodium Starch glycollate | extragranular | 12.5 mg | 5.0 |
| (Primojel) | | | |
| Magnesium stearate | extragranular | 2.5 mg | 1.0 |
| Total Tablet weight (uncoated) | | 250 mg | |

The process steps are as in Example 4 except that the steps 1.0 to 2.2 are altered:

1.0 Granulation.

1.1 Blend the SB-207266, microcrystalline cellulose, hydroxypropylmethyl cellulose and dibasic calcium phosphate dihydrate in a suitable high shear mixer-granulator for 5 mins with impeller at 200 rpm.
1.2 Add the purified water to effect the granulation as follows. Add 15% v/w=352.5 ml water over ca. 1.5-2 mins (pressure 1.2 bar, nozzle 1 mm 65°); turn chopper on; continue granulating for 5 mins to assess granulation; then add additional 5% v/w=117.5 ml water; then continue granulating until 9 mins.
1.2A Wet mill through a 0.375 inch screen with square leading edge impeller (150 spacer).
1.3 Dry the granules in a fluid bed drier at inlet temperature of 60° C.
1.4 Pass the dried granules through a 0.055 inch screen with 150 spacer and a square leading edge impeller using a comminuting mill, followed by screening through a 0.032 inch screen.
1.5 Determine the yield of the granules (80.7%).
   2.0 Manufacture of Compression Mix.
2.1 Blend the required quantity of sodium starch glycollate with the dried granules for 5 mins at 17 rpm in a 5 litre bin blender.
2.1A Add the required quantites of magnesium stearate with the product of 2.1 and blend for an additional 2 mins.
2.2 Determine the yield of compression mix.

### Example 7

The tablet of Example 6 containing 75 mg of SB 207266 (measured as free base) was modified by changing the percentage of HMPC binder from 2 weight % (5 mg/tablet) to 3 weight % (7.5 mg/tablet) by altering the calcium hydrogen phosphate from 38.7 wt % (96.75 mg/tablet) to 37.7 wt % (94.25 mg/tablet). The batch made was 2500 g dry weight again, for 10 tablets of 250 mg total weight. 20% v/w water for granulation was again used. The process was the same as for Example 6.

The particle size analysis of the granules produced by Examples 6 and 7 after milling but before blending with extragranular excipients and tabletting is shown in FIGS. 1 and 2 respectively. The equipment used was a Fritsch vibratory sieve shaker. The results, for 100.0 g of granules, are tabulated in the following Table 2:

TABLE 2
particle size of granules in 100 g batches from Examples 6 & 7

| | | | Example 7 (3 wt % HPMC) | |
|---|---|---|---|---|
| | Example 6 (2 wt % HPMC) | | | weight |
| Sieve | Weight in | weight % age | Weight in | % age |
| hole | grams retained | retained | grams retained | retained |
| size | on sieve (= %) | (cumulative) | on sieve (= %) | (cumulative) |
| BASE | 1.5 | 100 | 2.4 | 100.0 |
| 53 μm | 5.5 | 98.55 | 6.4 | 97.68 |
| 75 μm | 19.4 | 93.23 | 19.0 | 91.52 |
| 106 μm | 26.8 | 74.47 | 22.3 | 73.21 |
| 150 μm | 18.8 | 48.55 | 18.1 | 51.73 |
| 250 μm | 10.6 | 30.37 | 10.6 | 34.29 |
| 355 μm | 12.6 | 20.12 | 15.3 | 24.08 |
| 500 μm | 8.2 | 7.93 | 9.7 | 9.34 |

### Example 8

### Protocol for the Treatment or Prophylaxis of Atrial Fibrillation and/or Atrial Remodelling in Humans Using Orally Administered SB 207266

A proposed clinical protocol for the treatment or prophylaxis of atrial fibrillation and/or atrial remodelling using SB 207266 or a salt thereof is now described in detail.

This Protocol describes administration of SB 207266 or the salt (hereinafter “SB 207266”) to patients with symptomatic persistent atrial fibrillation (AF). The objective is the inhibition of symptomatic recurrences of atrial fibrillation in these patients with persistent AF. Patients with symptomatic persistent AF, of duration ≧48 hrs and <6 months, who require cardioversion (e.g. DC cardioversion) are suitable. Symptoms of persistent AF may for example include palpitations, etc. Patients preferably either have:
therapeutic anticoagulation (e.g. warfarin or coumarin) for ≧3 weeks before commencement of treatment, or
in the absence of therapeutic anticoagulation for ≧3 weeks, they have a transesophageal echocardiography (TEE) which is negative for clot and have received intravenous heparin until aPTT is stable and in the therapeutic range.

Patients receive SB 207266 preferably after such therapeutic anticoagulation, or after TEE in addition to iv heparin.

SB 207266 (e.g. as free base, but more preferably as the hydrochloride salt SB 207266-A) is generally administered at daily oral doses of 20 mg, 50 mg or 80 mg uid (measured as the free base). However, on day 1 of the administration of SB 207266, it is generally administered at a singl oral loading dose of 1.5 times (1.5×) the dosage allocated for the daily maintenance therapy. Therefore, preferably, a single oral loading dose of 30 mg, 75 mg or 120 mg is given on day 1, followed by a daily dose of 20 mg, 50 mg or 80 mg respectively on subsequent days.

The loading doses can be administered as three 10, 25 or 40 mg tablets given at the same time on day 1; the daily maintenance dose can be administered as two 10, 25 or 40 mg tablets given at the same time on subsequent days; the 10, 25 and 40 mg tablets used are preferably those described in Example 4 above.

About two or about five hours after administration of the first-day 1.5× oral loading dose of SB 207266, patients remaining in atrial fibrillation (and/or not pharmacologically cardioverted) preferably then undergo direct current (DC) cardioversion. Any of the following mono or bi-phasic cardioversion algorithms can be followed.

| | | Bi-Phasic | Bi-Phasic |
|---|---|---|---|
| Shock sequence | Mono-phasic | (option 1) | (option 2) |
| 1st Shock | 200 Joules | 170 Joules | 120 Joules |
| 2nd Shock | 250 Joules | 200 Joules | 150 Joules |
| 3rd Shock | 300 Joules | 230 Joules | 170 Joules |

If the patient does not revert to normal sinus rhythmn (NSR) after the 3rd shock using one of the above sequences the doctor may at his discretion proceed with further attempts at different energies. Successful cardioversion is defined as maintenance of NSR for ≧1 hr post-cardioversion.

Following a successful DC cardioversion to NSR, administration of SB 207266 to the patient can be continued once daily for 6 months (for example), or for shorter or longer periods. Those patients who spontaneously revert to normal sinus rhythmn (NSR) can also receive SB 207266 once daily for (e.g.) 6 months. Patients who experience a recurrence of AF during this daily treatment can be DC cardioverted back to sinus rhythm and can continue to receive SB 207266.

Patients should preferably continue on anticoagulation therapy (e.g. warfarin or coumarin) for at least the first four weeks following successful cardioversion, and more preferably throughout the period during which SB 207266 is administered.

The most preferred Protocol is therefore given below:

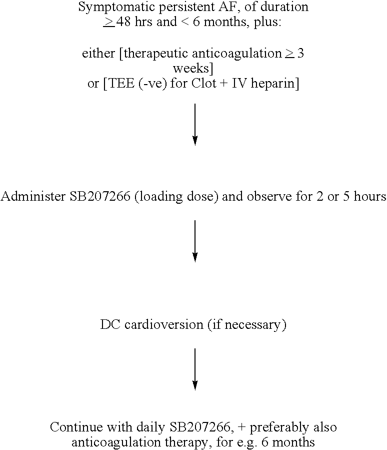

A “symptomatic recurrence” of AF includes or means an episode of palpitations or other symptoms typical for the patient. This can be further established by either a ECG (e.g. 12-lead ECG) recording showing evidence of atrial fibrillation or a rhythm strip recorded on a event recorder device and optionally reviewed by the doctor.

1. A pharmaceutical composition comprising the hydrochloride salt of N-[(1-ⁿbutyl-4-piperidinyl)methyl]-3,4-dihydro-2H-[1,3]oxazino[3,2-a]indole-10-carboxamide in combination with one or more pharmaceutically acceptable carriers, wherein at least some of the N-[(1-ⁿbutyl-4-piperidinyl)methyl]-3,4-dihydro-2H-[1,3]oxazino[3,2-a]indole-10-carboxamide hydrochloride salt is in granulated form,
and wherein the N-[(1-ⁿbutyl-4-piperidinyl)methyl]-3,4-dihydro-2H-[1,3]oxazino[3,2-a]indole-10-carboxamide hydrochloride salt is present in the composition in at least 4 weight % by weight of the composition.

2. A composition as claimed in claim 1 wherein substantially all of the N-[(1-ⁿbutyl-4-piperidinyl)methyl]-3,4-dihydro-2H-[1,3]oxazino[3,2-a]indole-10-carboxamide hydrochloride salt is in granulated form.

3. A composition as claimed in claim 1 wherein 50% or more by weight or by volume of the granules including the N-[(1-ⁿbutyl-4-piperidinyl)methyl]-3,4-dihydro-2H-[1,3]oxazino[3,2-a]indole-10-carboxamide hydrochloride salt have a particle size of ≧100 microns.

4. A composition as claimed in claim 1 wherein 50% or more by weight or by volume of the granules including the N-[(1-ⁿbutyl-4-piperidinyl)methyl]-3,4-dihydro-2H-[1,3]oxazino[3,2-a]indole-10-carboxamide hydrochloride salt have a particle size of ≧250 microns.

5. A composition as claimed in claim 1 wherein 50% or more by weight or by volume of the granules including the N-[(1-ⁿbutyl-4-piperidinyl)methyl]-3,4-dihydro-2H-[1,3]oxazino[3,2-a]indole-10-carboxamide hydrochloride salt have a particle size of 100 to 1000 microns.

6. A composition as claimed in claim 1 wherein 90% or more by weight or by volume of the granules including the N-[(1-ⁿbutyl-4-piperidinyl)methyl]-3,4dihydro-2H-[1,3]oxazino[3,2-a]indole-10-carboxamide hydrochloride salt have a particle size of ≧10 microns.

7. A composition as claimed in claim 1 wherein 90% or more by weight or by volume of the granules including the N-[(1-ⁿbutyl-4-piperidinyl)methyl]-3,4-dihydro-2H-[1,3]oxazino[3,2-a]indole-10-carboxamide hydrochloride salt have a particle size of ≧50 microns.

8. A composition as claimed in claim 1 wherein 50% or more by weight or by volume of the particles of the N-[(1-ⁿbutyl-4-piperidinyl)methyl]-3,4-dihydro-2H-[1,3]oxazino[3,2-a]indole-10-carboxamide hydrochloride salt have a particle size of ≦50 microns.

9. A composition as claimed in claim 1 wherein 10% or more by weight or by volume of the particles of the N-[(1-ⁿbutyl-4-piperidinyl)methyl]-3,4-dihydro-2H-[1,3]oxazino[3,2-a]indole-10-carboxamide hydrochloride salt have a particle size of ≦10 microns.

10. A composition as claimed in claim 1 wherein 50% or more by weight or by volume of the granules including the N-[(1-ⁿbutyl-4-piperidinyl)methyl]-3,4-dihydro-2H-[1,3]oxazino[3,2-a]indole-10-carboxamide hydrochloride salt have a particle size of ≧100 microns (micrometres); and wherein 10% or more by weight or by volume of the particles of the N-[(1-ⁿbutyl-4-piperidinyl)methyl]-3,4-dihydro-2H-[1,3]oxazino[3,2-a]indole-10-carboxamide hydrochloride salt have a particle size of ≦10 microns.

11. A composition as claimed in claim 1 wherein the N-[(1-ⁿbutyl-4-piperidinyl)methyl]-3,4-dihydro-2H-[1,3]oxazino[3,2-a]indole-10-carboxamide hydrochloride salt is present in the composition in at least 5 weight % by weight of the composition.

12. A composition as claimed in claim 1 wherein the N-[(1-ⁿbutyl-4-piperidinyl)methyl]-3,4-dihydro-2H-[1,3]oxazino[3,2-a]indole-10-carboxamide hydrochloride salt is present in the composition in at least 6 weight % by weight of the composition.

13. A composition as claimed in claim 1 wherein the hydrochloride salt of N-[(1-ⁿbutyl-4-piperidinyl)methyl]-3,4-dihydro-2H-[1,3]oxazino[3,2-a]indole-10-carboxamide is of a form obtainable by a process in which the N-[(1-ⁿbutyl-4-piperidinyl)methyl]-3,4-dihydro-2H-[1,3]oxazino[3,2-a]indole-10-carboxamide hydrochloride salt is dissolved in ethanol or an ethanol-containing solvent to form a solution and is crystallised from the solution by addition of a C₅-C₁₀ hydrocarbon and/or a solvent containing a C₅-C₁₀ hydrocarbon.

14. A composition as claimed in claim 1 wherein the granules containing the N-[(1-ⁿbutyl-4-piperidinyl)methyl]-3,4-dihydro-2H-[1,3]oxazino[3,2-a]indole-10-carboxamide hydrochloride salt also contain a filler/diluent.

15. A composition as claimed in claim 14 wherein the filler/diluent is abrasive.

16. A composition as claimed in claim 14 wherein the filler/diluent is insoluble, practically insoluble, very slightly soluble or slightly soluble in water and/or ethanol.

17. A composition as claimed in claim 14 wherein the filler/diluent is insoluble or practically insoluble in water and/or ethanol.

18. A composition as claimed in claim 14 wherein the filler comprises any pharmaceutically acceptable metal salt which is insoluble, practically insoluble, very slightly soluble or slightly soluble in water and/or ethanol.

19. A composition as claimed in claim 1 or 3 wherein the granules containing the N-[(1-ⁿbutyl-4-piperidinyl)methyl]-3,4-dihydro-2H-[1,3]oxazino[3,2-a]indole-10-carboxamide hydrochloride salt also contain a filler/diluent comprising CaHPO₄ and/or Ca₃(PO₄)₂.

20. A composition as claimed in claim 19, wherein the weight ratio of the filler to drug in the granules is at least 1:3, and wherein the filler is present in ≧15 weight % of the composition.

21. A composition as claimed in claim 16, wherein the filler is present in from 15 to 85% by weight of the composition.

22. A composition as claimed in claim 1 or 3 16 including a binder present in about 1 to about 10 weight % of the composition, and wherein the binder is soluble, freely soluble or very soluble in water.

23. A composition as claimed in claim 22 wherein the binder comprises hydroxypropylmethylcellulose (HPMC), hydroxypropylcellulose (HPC), hydroxyethylcellulose (HEC), hydroxymethylcellulose (HMC), methyl cellulose, ethyl cellulose, or povidone (polyvinylpyrollidone).

24. A composition as claimed in claim 1, 3 or 19 including an excipient which acts as a compression and/or granulation aid.

25. A composition as claimed in claim 1 being a tablet, or a capsule containing said composition.

26. A method of making a pharmaceutical composition comprising the hydrochloride salt of N-[(1-ⁿbutyl-4-piperidinyl)methyl]-3,4-dihydro-2H-[1,3]oxazino[3,2-a]indole-10-carboxamide in combination with one or more pharmaceutically acceptable carriers, wherein the N-[(1-ⁿbutyl-4-piperidinyl)methyl]-3,4-dihydro-2H-[1,3]oxazino[3,2-a]indole-10-carboxamide hydrochloride salt is present in the composition in at least 4 weight % by weight of the composition,
the method comprising forming at least some of the N-[(1-ⁿbutyl-4-piperidinyl)methyl]-3,4-dihydro-2H-[1,3]oxazino[3,2-a]indole-10-carboxamide hydrochloride salt into granules.

27. A method as claimed in claim 26 comprising mixing some or all of the N-[(1-ⁿbutyl-4-piperidinyl)methyl]-3,4-dihydro-2H-[1,3]oxazino[3,2-a]indole-10-carboxamide hydrochloride salt with a filler (diluent), and optionally a binder compression and/or granulation aid, before granulation.

28. A method as claimed in claim 27 wherein the granules are formed in the presence of a granulating solvent using a wet granulation process.

29. A method as claimed in claim 28 wherein the filler is insoluble, practically insoluble, very slightly soluble or slightly soluble in the granulation solvent.

30. A method as claimed in claim 29 wherein after formation the granules are milled to a particle size suitable for use in tablets or capsules.

31. A method as claimed in claim 26 wherein, after being formed and optionally milled, the granules are mixed with other pharmaceutically acceptable excipient(s) and compressed into tablets or filled into capsules.

32. A method of making a pharmaceutical composition comprising the hydrochloride salt of N-[(1-ⁿbutyl-4-piperidinyl)methyl]-3,4-dihydro-2H-[1,3]oxazino[3,2-a]indole-10-carboxamide in combination with one or more pharmaceutically acceptable carriers, wherein the N-[(1-ⁿbutyl-4-piperidinyl)methyl]-3,4-dihydro-2H-[1,3]oxazino[3,2-a]indole-10-carboxamide hydrochloride salt is present in the composition in at least 4 weight % by weight of the composition
the method comprising:
(a) dissolving the N-[(1-ⁿbutyl-4-piperidinyl)methyl]-3,4-dihydro-2H-[1,3]oxazino[3,2-a]indole-10-carboxamide hydrochloride salt in ethanol or an ethanol-containing solvent to form a solution,
(b) crystallising the N-[(1-ⁿbutyl-4-piperidinyl)methyl]-3,4-dihydro-2H-[1,3]oxazino[3,2-a]indole-10-carboxamide hydrochloride salt from the solution by addition of a C₅-C₁₀ hydrocarbon and/or a solvent containing a C₅-C₁₀ hydrocarbon, and
(c) forming at least some of the N-[(1-ⁿbutyl-4-piperidinyl)methyl]-3,4-dihydro-2H-[1,3]oxazino[3,2-a]indole-10-carboxamide hydrochloride salt into granules.

33. A method as claimed in claim 32 wherein 50% or more by weight or by volume of the granules including the hydrochloride salt of N-[(1-ⁿbutyl-4-piperidinyl)methyl]-3,4-dihydro-2H-[1,3]oxazino[3,2-a]indole-10-carboxamide have a particle size of ≧100 microns.

34. A method as claimed in claim 33 comprising the additional step after formation of the granules of (d) mixing the granules with other pharmaceutically acceptable excipient(s) and compressing into tablets or filling into capsules.

35. A composition as claimed in claim 1, wherein the HCl salt of N-[(1-ⁿbutyl-4-piperidinyl)methyl]-3,4-dihydro-2H-[1,3]oxazino[3,2-a]indole-10-carboxamide is of a form obtainable by a process in which the HCl salt is dissolved in ethanol or an ethanol-containing solvent to form a solution and is crystallised from the solution by addition of a C₅-C₁₀ hydrocarbon and/or a solvent containing a C₅-C₁₀ hydrocarbon.

36. A composition as claimed in claim 1, wherein the N-[(1-ⁿbutyl-4-piperidinyl)methyl]-3,4-dihydro-2H-[1,3]oxazino[3,2-a]indole-10-carboxamide or salt thereof is present in the composition in up to 70 weight % by weight of the composition.

37. A composition as claimed in claim 3, wherein the granules containing the N-[(1-ⁿbutyl-4-piperidinyl)methyl]-3,4-dihydro-2H-[1,3]oxazino[3,2-a]indole-10-carboxamide hydrochloride salt also contain a filler comprising CaHPO₄ and/or Ca₃(PO₄)₂, wherein the weight ratio of the filler to drug in the granules is at least 1:3, and wherein the filler is present in ≧15 weight % of the composition.

38. A composition as claimed in claim 1 wherein the granules containing the N-[(1-ⁿbutyl-4-piperidinyl)methyl]-3,4-dihydro-2H-[1,3]oxazino[3,2-a]indole-10-carboxamide hydrochloride salt also contain a filler comprising CaHPO₄.2H₂O.

39. A composition as claimed in claim 1 wherein the granules containing the N-[(1-ⁿbutyl-4-piperidinyl)methyl]-3,4-dihydro-2H-[1,3]oxazino[3,2-a]indole-10-carboxamide hydrochloride salt also contain a fine grade filler (diluent) being fine grade CaHPO₄ or fine grade Ca₃(PO₄)₂.

40. A composition as claimed in claim 24, wherein the compression and/or granulation aid is present inside the granules of the composition.

41. A composition as claimed in claim 40, wherein the compression and/or granulation aid is present in at least 15 weight % of the composition.

42. A composition as claimed in claim 41, wherein the compression and/or granulation aid is microcrystalline cellulose.

43. A composition as claimed in claim 24, wherein the compression and/or granulation aid is microcrystalline cellulose.

44. A composition as claimed in claim 1 or 19, wherein the composition includes a disintegrant present in about 1 to about 10 weight % of the composition and a lubricant present in about 0.2 to about 5 weight % of the composition.

45. A composition as claimed in claim 42, wherein the granules containing the N-[(1-ⁿbutyl-4-piperidinyl)methyl]-3,4-dihydro-2H-[1,3]oxazino[3,2-a]indole-10-carboxamide hydrochloride salt also contain a filler comprising CaHPO₄ and/or Ca₃(PO₄)₂, wherein the weight ratio of the filler to drug in the granules is at least 1:3, and wherein the filler is present in ≧15 weight % of the composition.

46. A composition as claimed in claim 45, wherein the composition includes a disintegrant present in about 1 to about 10 weight % of the composition and a lubricant present in about 0.2 to about 5 weight % of the composition.

47. A composition as claimed in claim 1, 3 or 19, wherein the intragranular ingredients form ≧70% by weight of the composition.

48. A composition as claimed in claim 45, wherein the intragranular ingredients form ≧70% by weight of the composition.

49. A composition as claimed in claim 1, 3, 16 or 19, wherein the granules have been formed in the presence of a granulating solvent using a wet granulation process.

50. A composition as claimed in claim 45, wherein the granules have been formed in the presence of a granulating solvent using a wet granulation process.

51. A method as claimed in claim 26 or 32 comprising:
mixing some or all of the N-[(1-ⁿbutyl-4-piperidinyl)methyl]-3,4-dihydro-2H-[1,3]oxazino[3,2-a]indole-10-carboxamide hydrochloride salt with a filler (diluent) comprising CaHPO₄ and/or Ca₃(PO₄)₂, and with microcrystalline cellulose, before granulation,
wherein the weight ratio of the filler to drug in the granules is at least 1:3, and wherein the filler is present in ≧15 weight % of the composition,
and wherein the microcrystalline cellulose is present in at least 15 weight % of the composition, and the microcrystalline cellulose is present inside the granules of the composition is intragranular but does not exclude that a portion is present outside the granules.
and wherein the granules are formed in the presence of a granulating solvent using a wet granulation process.
